(19) United States
(12) Patent Application Publication
(10) Pub. No.: US 2024/0293086 A1
(43) Pub. Date: Sep. 5, 2024

(54) METHOD AND SYSTEM TO QUANTIFY AND PREDICT CHANGES IN LUNG FUNCTION

(71) Applicant: ROYAL COLLEGE OF SURGEONS IN IRELAND, Dublin (IE)

(72) Inventors: Richard Costello, Dublin (IE); Garrett Greene, Dublin (IE)

(21) Appl. No.: 18/572,106

(22) PCT Filed: Jun. 22, 2022
(86) PCT No.: PCT/EP2022/067021

§ 371 Date: Dec. 19, 2023

(30) Foreign Application Priority Data
Jun. 22, 2021 (GB) 2108919.8

(51) Int. Cl. A61B 5/00; A61B 5/087; A61B 5/091; G16H 50/20

(52) U.S. Cl. CPC A61B 5/7275; A61B 5/0871; A61B 5/091; A61B 5/4833; A61B 5/4839; A61B 5/4848; A61B 5/4857; A61B 5/7267; G16H 50/20

(57) ABSTRACT

The present invention relates to a system and method to diagnose and predict an exacerbation for an individual, using electronically monitored expiratory lung function data and electronically monitored timing of inhalation of a bronchodilator. The method comprises the steps of computing a reference value or resting state value of at least one parameter indicating expiratory lung function of an individual, determining responsiveness of the individual to a bronchodilator using a non-linear time-series regression model, and estimation of amplitude and phase of the diurnal variation of the parameter for the individual using a regression analysis model. The reference value, responsiveness to bronchodilator, and the phase and amplitude of diurnal variation, are used to diagnose and predict occurrences of exacerbations for the individual.

### FIELD

The present disclosure relates to a method and system to quantify and predict changes in lung function, and more particularly to a system and method to diagnose and predict an exacerbation of an airways disease for an individual.

### BACKGROUND

An exacerbation of an airways disease such as asthma or COPD, that is, acute worsening of lung function, often requiring additional treatment is the largest contributor to healthcare costs in asthma and leads to a significant reduction in quality of life for patients. Therapy generally aims to reduce the rate of exacerbations; however, the effectiveness of treatment is hindered by poor understanding of the mechanisms leading to, or underlying exacerbations. Presently, since there is no widely agreed definition of an exacerbation and clinicians generally rely on patient self-reporting of symptoms to determine when an exacerbation is occurring.

Measurements of parameters such as Peak Expiratory Flow Rate (PEFR) or Forced Expiratory Volume in one second (FEV1) which indicate and quantify lung function, are commonly used to detect exacerbations exacerbation of an airways disease such as asthma or COPD. Since values of such parameters are known to vary widely between patients depending on age, height, sex, and other factors, such parameters are typically expressed in clinical use relative to a ‘reference’ or ‘expected’ value. In standard practice, the expected value of the parameter is calculated based on a patient's age, sex and height, and the recorded value of the parameter is expressed as a percentage of this expected value. This reference value or expected value is also employed by physicians while diagnosing an exacerbation. For example, physicians may consider the patient to be experiencing an exacerbation if the recorded FEV1 is less than 80% of the expected value. This method for diagnosing an exacerbation is known to be highly inaccurate due to the very wide variance in ‘normal’ lung function between patients with the same ‘expected’ value. For example, it is not uncommon for a healthy patient with no symptoms to consistently have a PEFR value of less than eighty percent of the expected value, and by contrast, for a patient having a typical PEFR of 110% of the expected value, a drop to even 90% of expected value can cause severe symptoms. Therefore, while this method may be appropriate at a population level, it is not appropriate at an individual level, as lung function parameter distribution is often highly skewed or multimodal.

Responsiveness of lung function to a bronchodilator is also an important diagnostic method known in the art for diagnosis of asthma. Responsiveness of lung function to a bronchodilator is typically assessed in a laboratory setting by performing a ‘reversibility’ test. In this test, spirometric measurements such as FEV1 or PEFR are first assessed using a lab spirometer.

A high dose of the bronchodilator is then administered, and spirometry is repeated after a fixed time period. The improvement in lung function measurement after the bronchodilator is administered, is used to quantify whether the patient has a “reversible” obstruction in airflow. In typical clinical practice, this is considered a pass/fail test, where the patient either displays or does not display a response to a bronchodilator. In reality however, responsiveness to bronchodilators varies greatly between patients, and may also depend on the current state of the patient's lung function. In particular, where the patient's lung function is already at or near its natural ‘healthy’ value, there may be little or no increase due to bronchodilator use. Therefore, this method does not give best results since it does not enable a quantitative estimate of responsiveness for a specific patient, for example a patient-specific value for the expected change in PEFR/FEV1 at a given time post bronchodilator use.

Expiratory Lung function in asthmatics is well known to display a diurnal variation linked to the patient's circadian rhythm, with values of PEFR/FEV1 typically being lower during the early morning and higher in the evening. The size or amplitude of this variation is known to increase during exacerbations and this feature is known in the art as a clinical feature for diagnosing exacerbation of an airways disease such as asthma or COPD.

The standard method for calculating diurnal variation is based on recording lung function parameters such as PEFR or FEV1 twice daily, either electronically or manually. The amplitude is calculated as the mean of the difference between ‘morning’ and ‘evening’ measurements on each day. This is then typically expressed as a percentage of the mean value, for example, a variation in PEFR between 450 L/min in the morning and 550 L/min in the evening might be expressed as

$$\frac{550 - 450}{( 550 + 450 ) / 2} \times 100 \% = 20 \% .$$

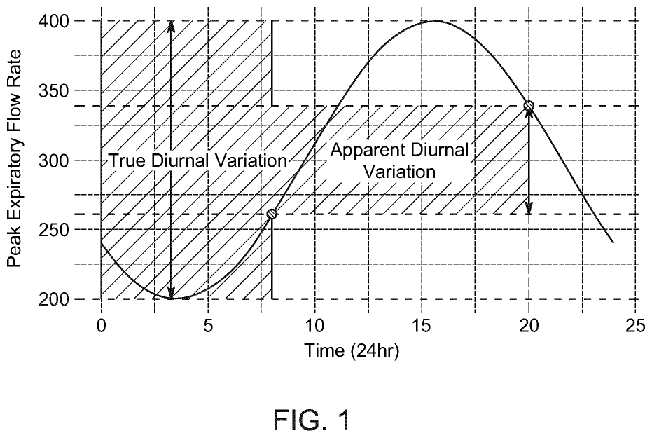

There are two obvious problems with this method. Firstly, it requires two lung function parameter measurements to be made each day, which is typically not complied with, since patients are generally irregular and may measure once a day or less frequently. Secondly, this method is based on an assumption that the ‘morning’ and ‘evening’ measurements correspond to the maximum and minimum values over the course of the day. In general, this is unlikely to be true as shown in FIG. 1 which illustrates the diurnal variation of a patient who measures his/her PEFR at 8 am and 8 pm, while the actual minimum and maximum PEFR values occur at 4 am and 4 pm respectively. Therefore, the measured ‘apparent diurnal variation’ is an underestimate of the ‘true diurnal variation’. Hence, the available methods for calculating the amplitude of diurnal variation are methodologically flawed and are highly unreliable and prone to bias. As a result, the use of diurnal variation as a diagnostic criterion for exacerbation of an airways disease such as asthma or COPD is no longer recommended.

There is therefore an unresolved and unfulfilled need in the art for a method to diagnose, detect, and predict exacerbations, which is individualized as per the lung function data and responsiveness to medication of each specific patient, and this forms the primary objective of the present invention.

### SUMMARY

The present invention relates to a system and method, as set out in the appended claims, to diagnose and predict an exacerbation for an individual, using electronically monitored expiratory lung function data of the individual and electronically monitored timing of usage of bronchodilator by the individual.

In a preferred embodiment of the present invention, a method to diagnose and predict an exacerbation, for example asthma, for an individual is provided. The method comprises the first step of computing a reference value for at least one measured value indicating expiratory lung function of the individual, wherein the reference value consists of the highest modal value with a density greater than a stated proportion of highest density in a distribution of the measured values estimated after a predetermined time duration subsequent to the timepoint at which the individual last inhaled a bronchodilator.

Further, the responsiveness of the individual to the inhaled bronchodilator is estimated by modelling the change in the measured values as a function of time using a non-linear time series regression model. The regression model has parameters comprising the decay rate of the bronchodilator, the rate of absorption of the bronchodilator, the bronchodilator responsiveness, dependence of bronchodilator response on current lung function; and one or more timepoints at which the bronchodilator was previously inhaled. Thereafter, the phase and amplitude of diurnal variation of the parameter is estimated using a regression analysis model and the measured values. A corrected value is then determined from the responsiveness of the individual to the inhaled bronchodilator, and the phase and amplitude of diurnal variation.

In an embodiment of the present invention, the corrected value is determined by subtracting the expected response of the individual to the inhaled bronchodilator and the instantaneous magnitude of the diurnal variation determined by the amplitude and phase calculated for the individual, from the instantaneous value of the parameter.

An exacerbation is diagnosed if the corrected value drops below a predetermined threshold relative to the reference value of the parameter for a predefined number of instances.

In an embodiment of the present invention, the predetermined threshold relative to the reference value for detecting exacerbation, is eighty percent of the reference value.

In an embodiment of the present invention, the predefined number of instances consists of two successive measurements.

Further, an exacerbation is predicted using the reference value, the responsiveness of the individual to the inhaled bronchodilator, the phase and amplitude of the diurnal variation of the individual, the corrected value, and timepoints of inhalation of the bronchodilator.

In an embodiment of the present invention, an exacerbation is predicted by applying the reference value, the responsiveness to inhaled bronchodilator, the phase and amplitude of the diurnal variation, the corrected value of the parameter, and timepoints of inhalation of the bronchodilator, to a time series regression model or a generalized regression model or a mixed effect regression model.

In an embodiment of the present invention, an exacerbation is predicted by applying the reference value, the responsiveness to inhaled bronchodilator, the phase and amplitude of the diurnal variation, the corrected value of the parameter, and timepoints of inhalation of the bronchodilator, to a supervised machine learning model.

In an embodiment of the present invention, the measured value is Peak Expiratory Flow Rate (PEFR).

In an embodiment of the present invention, the measured value is Forced Expiratory Volume in one second (FEV1).

In an embodiment of the present invention, the predetermined time duration is twice the half-life of the bronchodilator.

In an embodiment of the present invention, the regression analysis model for estimating the phase and amplitude of diurnal variation of the parameter, is a non-linear regression model based on a sinusoidal function.

In an embodiment of the present invention, the regression analysis model for estimating the phase and amplitude of diurnal variation of the parameter, is a weighted regression model using time-windowed functions.

In an embodiment of the present invention, the time windowed functions comprise a Gaussian window with a standard deviation determined by the expected time-scale of change in value of the parameter.

In an embodiment of the present invention, the highest modal value in the distribution of parameter values is estimated by kernel density estimation.

In an embodiment of the present invention, the bronchodilator comprises one of salbutamol/albuterol, formoterol, or salmeterol.

In an embodiment of the present invention, the parameter values and the timepoints of inhalation of the bronchodilator are determined using electronic means.

In a preferred embodiment of the present invention, a system to diagnose and predict an exacerbation for an individual is provided. The system comprises a computing device, a non-transitory memory means, at least one electronic handheld spirometer capable of measuring peak expiratory flow rate or FEV1, and an inhaler adherence monitor. The memory means, the spirometer, and the inhaler adherence monitor, are operably coupled to the computing device. The electronic peak expiratory flow meter is used to measure instantaneous values of lung function parameters such as PEFR and FEV 1, and the inhaler adherence monitor is used to measure the set of times at which the bronchodilator was used by the individual. The memory means has a plurality of instructions stored thereon which configures the computing device to compute a reference value for at least one measure value indicating expiratory lung function of the individual, the reference value consisting of the highest modal value having a density higher than a specified proportion of the highest density in a distribution of the values estimated after a predetermined time duration subsequent to the timepoint at which the individual last inhaled a bronchodilator; estimate the responsiveness of the individual to the inhaled bronchodilator by modelling the change in the parameter using a non-linear regression model, the regression model having variables comprising the decay rate of the bronchodilator, the rate of absorption of the bronchodilator, the variation in bronchodilator responsiveness, dependence of bronchodilator response on current lung function; and one or more historical timepoints at which the bronchodilator was inhaled; estimate the phase and amplitude of diurnal variation of the parameter using a regression analysis model; determine a corrected value of the parameter from the responsiveness of the individual to the inhaled bronchodilator and the phase and amplitude of the diurnal variation of the parameter; diagnose an exacerbation if the corrected value of the parameter drops below a predetermined threshold relative to the reference value of the parameter for a predefined number of instances; and predict an exacerbation from the reference value of the parameter, responsiveness to the inhaled bronchodilator, the phase and amplitude of the diurnal variation, the corrected value of the parameter estimated, and the one or more historical timepoints at which the bronchodilator was inhaled.

The present invention has wide industrial utility especially for insurance companies, pharmaceutical and healthcare companies, and medical device manufacturers such as manufacturers of smart inhalers, PEFR monitors, and manufacturers of medication.

This present invention can be used to personalise treatment by identifying patients most at risk for acute exacerbations and deploying healthcare resources appropriately. In addition, the present invention can potentially be used as part of a system to prevent or control acute asthma symptoms by adjusting treatment in real time based on the early detection of an incipient exacerbation. The present invention hence significantly reduces costs for treatment of exacerbation, for example by avoiding hospitalization for patients in most cases. The present invention also avoids side effects and related medical conditions by obviating the need for patients to use antibiotics/oral corticosteroids.

The present invention utilizes electronic data on lung function and timing of bronchodilator use to detect or predict exacerbations. This is extremely important since many asthma patients are heavily reliant on inhaler use to sustain good lung function, which may mask the features of exacerbations when considering lung function in isolation. Estimating personal characteristics including diurnal variation of lung function and responsiveness to inhaled therapy, and using these inputs to correct lung function measurements, enables the present invention to personalize prediction of an exacerbation using a patient's own data. This helps in prevention or mitigation of exacerbations through early detection and intervention.

The present invention overcomes the difficulty of objectively identifying and classifying exacerbations. Presently both clinical studies and asthma treatment guidelines generally define exacerbations through medication use or through self-reporting of symptoms. These methods are clearly susceptible to a number of confounding factors, not least differences in patient behaviour and perception of symptom severity. Furthermore, these definitions can only be applied retrospectively, and so are of little use for prevention and mitigation of exacerbations. The present invention predicts and identifies exacerbations at an early stage through objectively defined physiological criteria, and can potentially be applied in “real-time”, allowing for adjustment of treatment to prevent/control exacerbations.

The present invention hence provides a robust solution to problems identified in the art. Other advantages and additional novel features of the present invention will become apparent from the subsequent detailed description.

### DETAILED DESCRIPTION OF DRAWINGS

The present invention will be more clearly understood from the following description of an embodiment thereof, given by way of example only, with reference to the accompanying drawings, in which:—

FIG. 1 is a graphical representation illustrating estimation of diurnal variation using prior art methods.

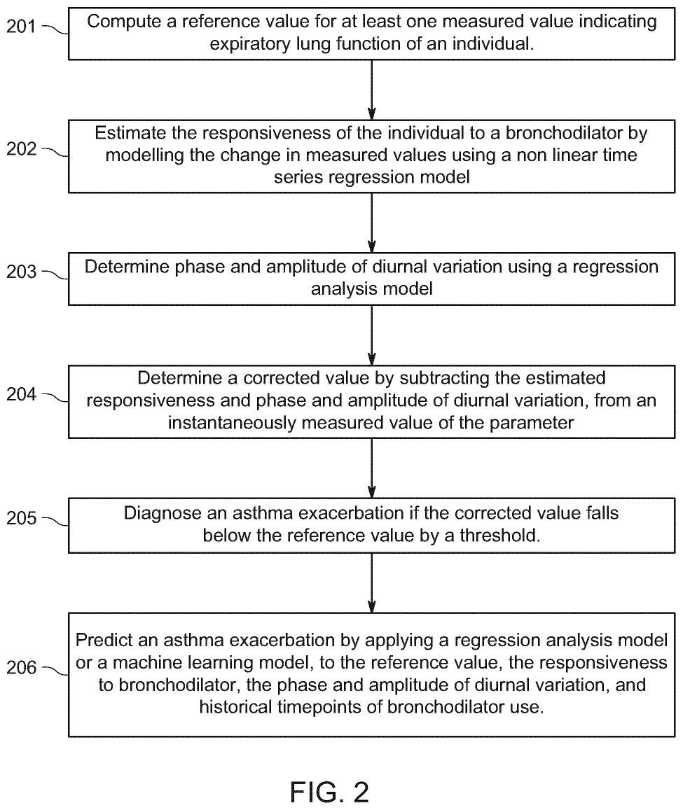

FIG. 2 is a flow diagram illustrating a method as per a preferred embodiment of the present invention.

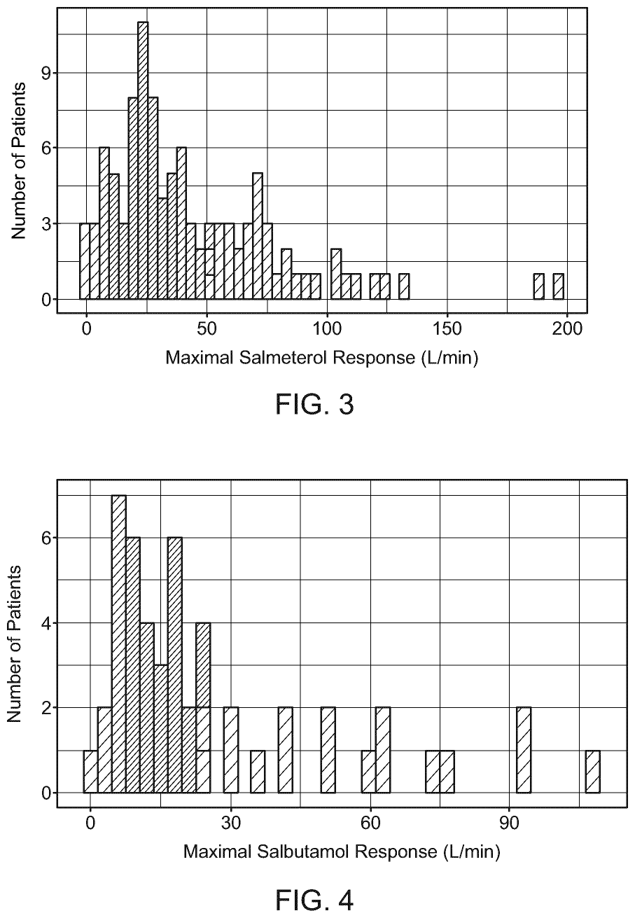

FIG. 3 is a graphical representation illustrating variance in responsiveness of a group of patients to Salmeterol, as per a preferred embodiment of the present invention.

FIG. 4 is a graphical representation illustrating variance in responsiveness of a group of patients to Salbutamol, as per a preferred embodiment of the present invention.

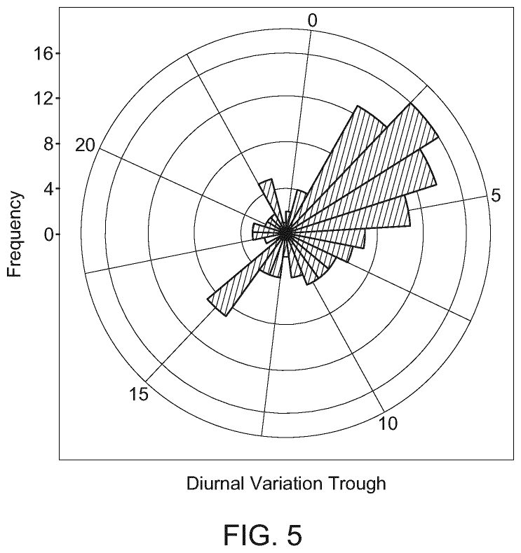

FIG. 5 is a circular histogram illustrating the distribution of diurnal variation trough times for a set of individuals.

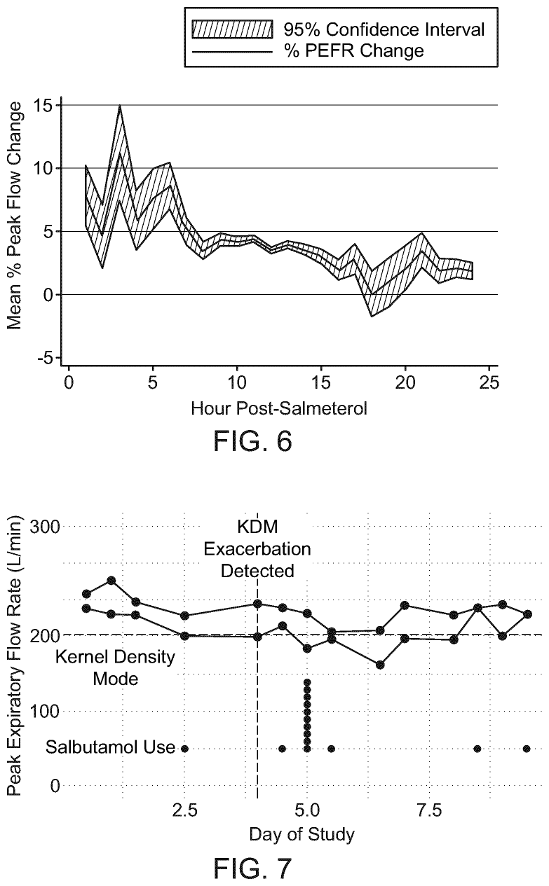

FIG. 6 is a graphical representation illustrating the variation in mean percentage of PEFR and 95% confidence interval of PEFR, with time following use of Salmeterol, as per a preferred embodiment of the present invention.

FIG. 7 is a graphical representation illustrating detection of an exacerbation from the corrected value of the lung function parameter, as per a preferred embodiment of the present invention.

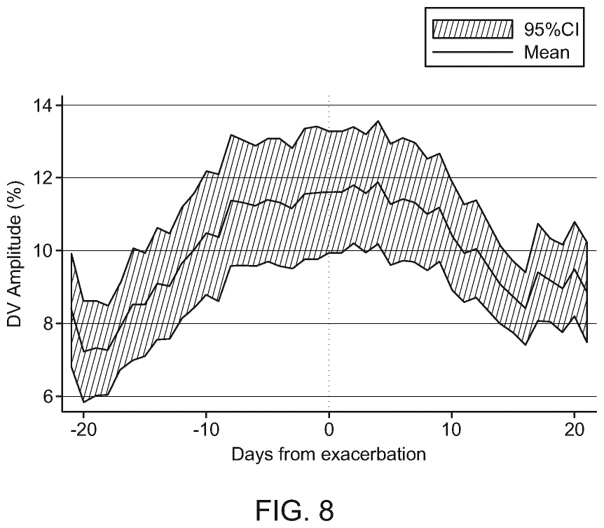

FIG. 8 illustrates the capacity of the method to predict exacerbations according to one embodiment of the present invention.

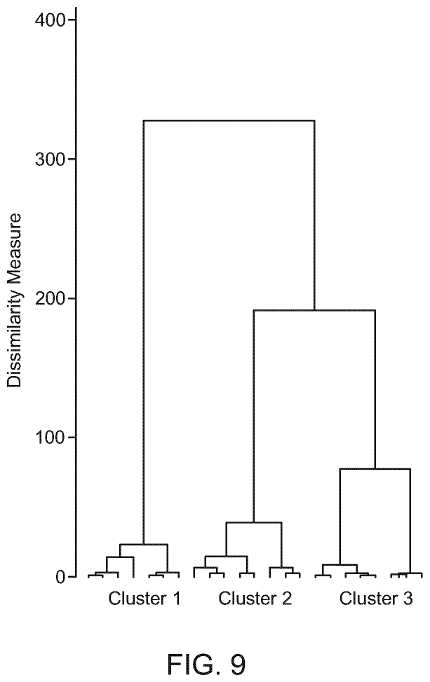

FIG. 9 is a graphical representation illustrating the dissimilarity between clusters of exacerbations ascertained using the present invention.

### DETAILED DESCRIPTION OF DRAWINGS

FIG. 2 is a flow diagram illustrating a method as per a preferred embodiment of the present invention. The method comprises the first step of computing a reference value or resting state value for at least one measured value indicating expiratory lung function, such as Peak Expiratory Flow Rate (PEFR) and Forced Expiratory Volume in one second (FEV1). The instantaneous values of the one or more measured values are electronically measured using a peak expiratory flow meter. The reference value for an individual is the modal value of the distribution of the parameter values estimated by Kernel density estimation over all recorded values for which the time since the last bronchodilator usage is greater than 2t_{1/2}, where t_{1/2} is the half-life of the bronchodilator used, such as salbutamol/albuterol or salmeterol or formoterol. In the case where more than one mode is found, the reference value is defined as the value of the highest mode, that is the mode whose lung function value is the highest and whose density is higher than a specified proportion of the density of the largest mode, that is the mode with the largest density. The historical timepoints at which a bronchodilator is used by the individual, is determined using an inhaler adherence monitor. The responsiveness of the individual to the bronchodilator 202, and the phase and amplitude of the diurnal variation of the parameter is then estimated 203.

To estimate responsiveness of the individual to the bronchodilator, the change in the measured value due to use of the bronchodilator is modelled as a function of time since previous bronchodilator use by a non-linear time-series regression model. This model takes into account one or more parameters the decaying time-course of the bronchodilator effect, the variation in bronchodilator responsiveness, and the dependence of bronchodilator response on current lung function. The inputs to this model are fitted based on each individual's data to give a quantitative estimate of responsiveness for a specific individual, that is the value for the expected change in the lung function parameter of the individual, at a given time post bronchodilator use. This is important since the responsiveness to a bronchodilator by different individuals widely varies as illustrated in FIG. 3 and FIG. 4. FIG. 3 illustrates the variance in responsiveness of a group of individuals to Salmeterol, and FIG. 4 illustrates the variance in responsiveness of a group of individuals to Salbutamol.

The non-linear regression model is hence personalised to characterise the bronchodilator's effect on the value of the parameter for each individual.

The estimated value of responsiveness may be of use in itself as a diagnostic measure and may also be used to give improved estimates of current value of the parameter by removing the masking effect of heavy bronchodilator use.

The responsiveness of the individual to the bronchodilator can be modelled as a simple decaying exponential function. The time between each variable reading and the respective previous doses of the bronchodilator were calculated, and the active doses were calculated using the bronchodilator's decay rate. For a parameter measurement taken at time t:

$${L}_{measured} ( t ) = {L}_{corrected} ( t ) + δ {\sum }_{{t}_{b} < t} {( t - {t}_{b} )}^{p} {e}^{- α ( t - {t}_{b} )} + ϵ \qquad ( 1 )$$

Where L_{measured}(t) is the value of the lung function measured at time t; L_{corrected}(t) is the ‘true’ underlying lung function, that is the value which would be measured if no bronchodilator was used by the individual; δ≥0 is the responsiveness to the bronchodilator of the individual in question; and ϵ is a random noise term. The parameter p controls the shape of the bronchodilator absorption curve, while a represents the decay rate of the bronchodilator. α is inversely proportional to the half-life of the bronchodilator drug. For example, the half-life for salbutamol and salmeterol is 4 hours and 11 hours respectively, corresponding to relatively larger and smaller values of a. a and p may be treated as model parameters, or fixed based on the available pharmacodynamic data for the bronchodilator in question. In an embodiment of the present invention, the exponent p is set to zero, giving a model with instantaneous absorption and exponential decay of the absorbed drug. The set of times (t_{b}) represent the times at which the bronchodilator was used as recorded by the inhaler adherence monitor.

If L_{corrected}(t) is assumed to be approximately constant over the period between two measurements at times t₁, t₂, the change in lung function is given by:

$$Δ {L}_{measured} = δ ( {\sum }_{{t}_{b} < {t}_{2}} {( {t}_{2} - {t}_{b} )}^{p} {e}^{- α ( {t}_{2} - {t}_{b} )} - {\sum }_{{t}_{b} < {t}_{1}} {( {t}_{1} - {t}_{b} )}^{p} {e}^{- α ( {t}_{1} - {t}_{b} )} ) + ϵ$$

In another embodiment of the present invention, the lung function may be modelled as:

$${L}_{measured} ( t ) = {L}_{corrected} ( t ) + δ f ( {L}_{measured} ) {\sum }_{{t}_{b} < t} {e}^{- α ( t - {t}_{b} )} + ϵ \qquad ( 2 )$$

Where f(L_{measured}) is a function having the property that it takes a value close to 1 when L_{measured} is significantly less than a suitable reference value and approaches 0 when L_{measured} is significantly greater than the reference value.

For example, f( ) may take the form of a sigmoid function:

$$f ( {L}_{measured} ) = \frac{1}{1 + {e}^{b ( {L}_{measured} - {L}_{ref} )}}$$

Where b is a positive constant and L_{ref} is the reference value of the parameter of the individual computed as described above. The difference between two measurements, assuming Lₜᵣᵤₑ(t) is constant, is then given by.

$$Δ {L}_{measured} = δ f ( {L}_{measured} ) ( {\sum }_{{t}_{b} < {t}_{2}} {( {t}_{2} - {t}_{b} )}^{p} {e}^{- α ( {t}_{2} - {t}_{b} )} - {\sum }_{{t}_{b} < {t}_{1}} {( {t}_{1} - {t}_{b} )}^{p} {e}^{- α ( {t}_{1} - {t}_{b} )} ) + ϵ$$

In equations (1) and (2), the aim is to obtain an estimate of the value of parameter δ, that is the responsiveness of the individual to the inhaled bronchodilator. In both cases, an estimate of this value is obtained by fitting a generalized statistical regression model, either using the formulae (1) or (2), or where L_{corrected}(t) is assumed approximately constant, using the formulae for the difference between measurements.

The diurnal variation for an individual is estimated by modelling the variation as a sinusoidal function in time with the individual specific amplitude and phase. This can be mathematically represented as:

$${L}_{measured} ( t ) = {L}_{0} + A * sin ( 2 π t + ϕ ) + ϵ = {L}_{0} + A ( \begin{matrix}sin ( 2 π t ) cos ( ϕ ) + \\ cos ( 2 π t ) sin ( ϕ )\end{matrix} ) + ϵ \qquad ( 3 )$$

Where ‘t’ is the time in days, A is the amplitude of diurnal variation, ϕ is the phase shift of the sinewave relative to time, L₀ is the underlying value of the parameter around which it exhibits a sinusoidal variation, and ϵ is a random noise term. The amplitude ‘A’ corresponds to the difference in the parameter value on the sine wave from its baseline to either its peak or its trough. The diurnal variation in the value of the parameter that an individual exhibits throughout a day is equal to the difference between the peak and trough value and is therefore equal to twice the amplitude value. The parameters A and ϕ, are individual-specific parameters to be fitted from the data by generalised linear regression, or non-linear least squares regression methods. For comparison with traditional methods, the diurnal variation can be expressed as:

$$DV = \frac{2 \vert A \vert}{{L}_{ref}} \times 100 \%$$

Where L_{ref} is a patient specific healthy reference value of the parameter. The inputs to the model are specific to an individual to personalise the measurement of each individual's diurnal variation. FIG. 5 illustrates the distribution of diurnal variation trough times for a set of individuals, with the modal value occurring at approximately 03:30, and broad variation around this value. This demonstrates the need to know precisely when the parameter was recorded.

In an embodiment of the present invention, the amplitude and phase may also vary with time, and are estimated by weighted regression using time-windowed functions, for example, a Gaussian window with a standard deviation determined by the expected time-scale of change in the value of the parameter.

In an embodiment of the present invention, the regression model used to estimate the individual's responsiveness to the bronchodilator as described in equations (1) and (2) above, may first be applied and used to estimate L_{corrected}(t). The diurnal variation model may then be fitted using the corrected value of the parameter L_{corrected}(t) rather than the instantaneously measured value of the parameter, L_{measured}(t).

In an embodiment of the present invention, the responsiveness of the individual to the bronchodilator, and the phase and amplitude of diurnal variation of the parameter may be fitted simultaneously in a combined model as provided below:

$${L}_{measured} ( t ) = {L}_{corrected} + δ f ( {L}_{measured} ) {\sum }_{{t}_{b} < t} {e}^{- α ( t - {t}_{b} )} + A * sin ( 2 π t + ϕ ) + ϵ \qquad ( 4 )$$

Each individual's lung function parameter value is then corrected to account for diurnal variations and the responsiveness and timing of inhaler use 204. The bronchodilator responsiveness, δ, and the time-varying amplitude and phase of diurnal variation, is used to obtain a corrected value or ‘true’ underlying value of the lung function parameter by rearranging equation (4), and allowing for A and ϕ to vary in time:

$${L}_{corrected} ( t ) = {L}_{measured} - δ f ( {L}_{measured} ) {\sum }_{{t}_{b} < t} {e}^{- α ( t - {t}_{b} )} - A ( t ) * sin ( 2 π t + ϕ ( t ) ) + ϵ \qquad ( 5 )$$

This corrected value of the lung function parameter gives a more accurate representation of lung health. In particular, the corrected value of lung function allows for the detection of changes in lung function which were otherwise masked by bronchodilator use or irregularity in the timing of lung function parameter measurements. FIG. 6 illustrates the percentage mean change in PEFR and change in the 95% confidence interval of PEFR with time (in hours) subsequent to administration of Salmeterol. FIG. 6 sufficiently demonstrates that use of bronchodilators prior to lung function parameter measurement can artificially inflate the parameter value. Hence, an accurate value or true value of a lung function parameter of an individual can only be obtained if aspects such as timing of bronchodilator use and responsiveness to bronchodilator are factored in. In one embodiment determining a corrected value of the parameter can be obtained by subtracting the instantaneous bronchodilator response estimated and the estimated instantaneous diurnal variation.

An exacerbation is detected and diagnosed if the corrected value of the parameter drops or falls below a predetermined threshold relative to the reference value of the lung function parameter on a predefined number of instances 205.

For example, one can define an exacerbation as any period during which L_{corrected}(t)<0.8*L_{ref} on two successive measurements, wherein L_{ref} is the reference value of the lung parameter or the highest kernel density mode of the lung function parameter distribution as described above.

FIG. 7 illustrates how the corrected values of the lung function parameter allows detection of an exacerbation which would otherwise be masked in the uncorrected value of the instantaneous measurement of the lung function parameter inflated by use of a bronchodilator such as Salbutamol. The representation in blue in FIG. 7 indicates the corrected parameter values, the representation indicates the uncorrected parameter values or the instantaneous measured values of the parameter inflated by bronchodilator use, and the representation indicates the critical value, of the parameter, in this case 0.8*L_{ref} representing the lower limit of good health.

The values of the reference value for an individual (L_{ref}), the responsiveness to bronchodilator (δ), the phase and amplitude of diurnal variation, the corrected value of the parameter ((L_{corrected}), and one or more historical timepoints for bronchodilator use can be used to predict a future occurrence of exacerbation in an individual through statistical features of said data 206. This is achieved through a combination of standard statistical techniques, such as generalised linear models, or mixed effects regression; or through supervised machine learning approaches.

FIG. 8 illustrates the capacity of the method to predict exacerbations. The Diurnal variation amplitude was estimated in a continuously time-windowed fashion. FIG. 8 displays the mean DV amplitude estimated by time-windowed non-linear regression in the three weeks before and after physician diagnosis of exacerbations. Mean and 95% CI for DV are calculated from PEFR recordings of 101 reported exacerbations in 55 distinct patients. The DV amplitude is significantly elevated compared to baseline up to 10 days prior to diagnosis. Elevated DV is a significantly predictive marker of incipient exacerbation.

The present invention enables stratification of individuals into one or more risk groups based on risk factors for exacerbation. The present invention further enables identification of distinct exacerbation phenotypes and clusters exacerbations into three distinct types—a loss of control exacerbation, a sudden lung function parameter (for example PEFR) drop exacerbation, and a non-lung function parameter exacerbation.

These different types of exacerbation may require different treatment approaches, and so identification of these types further contributes to personalisation of treatment. The different types of exacerbation are characterised by differences in the pattern of lung function variation and medication use. FIG. 9 illustrates the dissimilarity between different clusters of exacerbations ascertained using the present invention.

In a preferred embodiment of the present invention, a system for diagnosing and predicting an exacerbation is provided. The system comprises a computing device, a non-transitory memory means, at least one electronic peak expiratory flow meter, and an inhaler adherence motor. The memory means, the electronic peak expiratory flow meter, and the inhaler adherence monitor, are operably coupled to the computing device. The electronic peak expiratory flow meter is used to measure instantaneous value of lung function such as PEFR and FEV 1, and the inhaler adherence motor is used to measure the set of times at which the bronchodilator was used by the individual.

The memory means may be any internal or external device or web-based data storage mechanism adapted to store data. The computing device may be a personal computer, a portable device such as a tablet computer, a laptop, a smart phone, connected medical device or any operating system based connected portable device.

The memory means has a plurality of instructions stored thereon which configures the computing device to: compute a reference value for at least one measured value, such as PEFR or FEV1, indicating expiratory lung function of the individual, the reference value consisting the highest modal value with the highest density in a distribution of the parameter values estimated after a predetermined time duration subsequent to the timepoint at which the individual last inhaled a bronchodilator such as salbutamol or salmeterol; estimate the responsiveness of the individual to the inhaled bronchodilator by modelling the change in the measured values using a non-linear time series regression model, the regression model having variables comprising the decay rate of the bronchodilator, the rate of absorption of the bronchodilator, the bronchodilator responsiveness, dependence of bronchodilator response on current lung function; and one or more historical timepoints at which the bronchodilator was inhaled; estimate the phase and amplitude of diurnal variation of the parameter using a regression analysis model; determine a corrected value of the parameter from the responsiveness of the individual to the inhaled bronchodilator and the phase and amplitude of the diurnal variation of the parameter; diagnose an exacerbation if the corrected value drops below a predetermined threshold relative to the reference value for a predefined number of instances, for example an exacerbation is diagnosed if the corrected value of the parameter drops below eighty percent of the reference value on two successive measurements; and predict an exacerbation from the reference value, responsiveness to the inhaled bronchodilator, the phase and amplitude of the diurnal variation, the determined corrected value of the parameter, and the one or more historical timepoints at which the bronchodilator was inhaled.

In an embodiment of the present invention, predetermined time duration is twice the half-life of the bronchodilator.

It will be appreciated that in the context of the present invention a system and method to predict exacerbations is described, the invention can also be used to predict exacerbations of Chronic Obstructive Pulmonary Disease (COPD) using the system and method described herein.

In one embodiment there is provided a computer implemented method to diagnose and predict a Chronic Obstructive Pulmonary Disease (COPD) exacerbation for an individual, the method comprising the steps of computing a reference value for at least one parameter indicating expiratory lung function of the individual, the reference value consisting the highest modal value with the highest density in a distribution of the parameter values determined after a predetermined time duration subsequent to the timepoint at which the individual last inhaled a bronchodilator The responsiveness of the individual to the bronchodilator is estimated by modelling the change in the values using a non-linear time series regression model, the regression model having parameters comprising the decay rate of the bronchodilator, the rate of absorption of the bronchodilator, the variation in bronchodilator responsiveness, dependence of bronchodilator response on current lung function; and one or more timepoints at which the bronchodilator was previously inhaled. The phase and amplitude of diurnal variation of the parameter are estimated using a regression analysis model. The system and method further performs the steps of determining a corrected value from the responsiveness estimated and the phase and amplitude of the diurnal variation estimated in step.

A Chronic Obstructive Pulmonary Disease (COPD) exacerbation can be diagnosed, if the corrected value drops below a predetermined threshold relative to the reference value computed in step, for a predefined number of instances. The Chronic Obstructive Pulmonary Disease (COPD) exacerbation can be predicted from the reference value computed, responsiveness estimated, the phase and amplitude of the diurnal variation estimated, the corrected value of the parameter estimated in step, and one or more historical timepoints of inhalation of the bronchodilator.

Although the present invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present invention as defined.

Further, a person ordinarily skilled in the art will appreciate that the various illustrative method steps described in connection with the embodiments disclosed herein may be implemented using electronic hardware, or a combination of hardware and software. To clearly illustrate this interchangeability of hardware and a combination of hardware and software, various illustrations and steps have been described above, generally in terms of their functionality. Whether such functionality is implemented as hardware or a combination of hardware and software depends upon the design choice of a person ordinarily skilled in the art. Such skilled artisans may implement the described functionality in varying ways for each particular application, but such obvious design choices should not be interpreted as causing a departure from the scope of the present invention.

In the specification, the terms “comprise, comprises, comprised and comprising” or any variation thereof and the terms “include, includes, included and including” or any variation thereof are considered to be totally interchangeable, and they should all be afforded the widest possible interpretation and vice versa.

1. A computer implemented method to diagnose and predict an exacerbation for an individual, the method comprising the steps of:
a) computing a reference value for at least one measured value indicating expiratory lung function of the individual, the reference value consisting the highest modal value with the highest density in a distribution of the measured values determined after a predetermined time duration subsequent to the timepoint at which the individual last inhaled a bronchodilator;
b) estimating the responsiveness of the individual to the bronchodilator by modelling the change in the measured values using a non-linear time series regression model, the regression model having parameters comprising at least two or more of the following: the decay rate of the bronchodilator, the rate of absorption of the bronchodilator, the bronchodilator responsiveness, dependence of bronchodilator response on current lung function; and one or more timepoints at which the bronchodilator was previously inhaled;
c) estimating the phase and amplitude of diurnal variation of the parameter using a regression analysis model and the measured values;
d) determining a corrected value from the responsiveness estimated in step (b), and the phase and amplitude of the diurnal variation estimated in step (c);
e) diagnosing an exacerbation if the corrected value drops below a predetermined threshold relative to the reference value computed in step (a), for a predefined number of instances; and/or
f) predicting an exacerbation from the reference value computed in step (a), responsiveness estimated in step (b), the phase and amplitude of the diurnal variation estimated in step (c), the corrected value estimated in step (d), and one or more historical timepoints of inhalation of the bronchodilator.

2. The method as claimed in claim 1, wherein the measured value is Peak Expiratory Flow Rate.

3. The method as claimed in claim 1, wherein the measured value is Forced Expiratory Volume in one second (FEV1).

4. The method as claimed in any preceding claim wherein the regression analysis model for estimating the phase and amplitude of diurnal variation of the parameter, is a non-linear time series regression model based on a sinusoidal function.

5. The method as claimed in any of claims 1 to 3, wherein the regression analysis model for estimating the phase and amplitude of diurnal variation of the parameter, is a weighted regression model using time-windowed functions.

6. The method as claimed in claim 5, wherein the time windowed functions comprise a Gaussian window with a standard deviation determined by the expected time-scale of change in value of the parameter.

7. The method as claimed in any of the preceding claims wherein the highest modal value in the distribution of parameter values is determined by kernel density estimation.

8. The method as claimed in any of the preceding claims, wherein the predetermined time duration is twice the half-life of the bronchodilator.

9. The method as claimed in any of the preceding claims, further comprising the step of stratifying a plurality of individuals into one or more groups based on risk factors for exacerbation.

10. The method as claimed in any of the preceding claims, wherein the bronchodilator comprises one of salbutamol/albuterol, formoterol, or salmeterol.

11. The method as claimed in any of the preceding claims, wherein the step of prediction of exacerbation is performed by applying the reference value, the responsiveness to the bronchodilator, the phase and amplitude of the diurnal variation, the corrected value of the parameter, and timepoints of inhalation of the bronchodilator, to one of, a time series regression model or a generalized regression model or a mixed effect regression model.

12. The method as claimed in any of the preceding claims, wherein the step of prediction of exacerbation is performed by applying the reference value, the responsiveness to the bronchodilator, the phase and amplitude of the diurnal variation, the corrected value of the parameter, and timepoints of inhalation of the bronchodilator, to a supervised machine learning model.

13. The method as claimed in any of the preceding claims, wherein the predetermined threshold relative to the reference value for detecting exacerbation, is eighty percent of the reference value.

14. The method as claimed in any of the preceding claims, wherein the predefined number of instances consists of two successive measurements.

15. The method as claimed in any of the preceding claims, wherein the corrected value is determined by subtracting the response of the individual to the bronchodilator and the phase and amplitude of the diurnal variation of the parameter, from the instantaneous value of the parameter.

16. The method as claimed in any of the preceding claims wherein the measured values and the timepoints of inhalation of the bronchodilator are determined using electronic means.

17. A system to diagnose and predict an exacerbation for an individual,
the system comprising:
a computing device;
a non-transitory memory means operably coupled to the computing device;
at least one electronic handheld spirometer operably coupled to the computing device;
and an inhaler adherence monitor operably coupled to the computing device;
the memory means has a plurality of instructions stored thereon which configures the computing device to:
compute a reference value for at least one measured value indicating expiratory lung function of the individual, the reference value consisting the highest modal value with the highest density in a distribution of the measured values estimated after a predetermined time duration subsequent to the timepoint at which the individual last inhaled a bronchodilator;
estimate the responsiveness of the individual to the bronchodilator by modelling change in the measured values using a non-linear time series regression model, the regression model having parameters comprising at least two or more of the following: the decay rate of the bronchodilator, the rate of absorption of the bronchodilator, bronchodilator responsiveness, dependence of bronchodilator response on current lung function; and one or more historical timepoints at which the bronchodilator was inhaled;
estimate the phase and amplitude of diurnal variation of the parameter using a regression analysis model and the measured values;
determine a corrected value from the responsiveness of the individual to the bronchodilator and the phase and amplitude of the diurnal variation of the parameter;
diagnose an exacerbation if the corrected value drops below a predetermined threshold relative to the reference value of the parameter for a predefined number of instances; and/or
predict an exacerbation from the reference value of the parameter, responsiveness to the bronchodilator, the phase and amplitude of the diurnal variation, the estimated corrected value of the parameter, and the one or more historical timepoints at which the bronchodilator was inhaled.

18. The system as claimed in claim 17, wherein the measured value is Peak Expiratory Flow Rate.

19. The system as claimed in claim 17, wherein the measured value is Forced Expiratory Volume in one second (FEV1).

20. The system as claimed in any of claims 17 to 19 wherein the predetermined time duration is twice the half-life of the bronchodilator.

21. The system as claimed in any of any of claims 17 to 20, wherein the computed device is configured to stratify a plurality of individuals into one or more groups based on risk factors for exacerbation.

22. The system as claimed in any of claims 17 to 21, wherein the bronchodilator comprises one of salbutamol/albuterol, formoterol, or salmeterol.

23. The system as claimed in any of claims 17 to 22, wherein the predetermined threshold relative to the reference value for detecting exacerbation, is eighty percent of the reference value.

24. The system as claimed in any of claims 17 to 23, wherein the predefined number of instances consists of two successive measurements.
